(19) United States
(12) Patent Application Publication
(10) Pub. No.: US 2002/0183530 A1
(43) Pub. Date: Dec. 5, 2002

(54) Method for purifying phytosterol concentrates using a pressurized fluid (carbon dioxide)

(75) Inventors: Olli Aaltonen, Helsinki (FI); Sampsa Haarasilta, Kerava (FI); Pekka Piiroinen, Kirkkonummi (FI); Markku Rantakyla, Helsinki (FI); Jouko Virtanen, Nastola (FI); Antti Kaipainen, Espoo (FI)

Correspondence Address: SCULLY, SCOTT, MURPHY & PRESSER, 400 Garden City Plaza, Garden City, NY, 11530, US

(73) Assignee: DANISCO SUGAR OY, ESPOO (FI)

(21) Appl. No.: 10/038,211

(22) Filed: Jan. 4, 2002

Related U.S. Application Data
(63) Continuation of application No. PCT/FI00/00612, filed on Jul. 4, 2000.

(30) Foreign Application Priority Data
Jul. 5, 1999 (FI) 991533

(51) Int. Cl. C07J 9/00

(52) U.S. Cl. 552/545

(57) ABSTRACT

The invention relates to a method of purifying phytosterol concentrates by contacting the phytosterol concentrate with a pressurized fluid. The invention also relates to the obtained purified phytosterol concentrate, foodstuff and animal feed premixes containing the same as well as to foodstuffs and animal feeds containing phytosterol concentrate.

This is a continuation of PCT application No. PCT/FI00/00612 Dated Jul. 4, 2000

### BACKGROUND OF THE INVENTION

The invention relates to a method of purifying phytosterol concentrates by treating the sterol concentrate with a pressurized fluid, typically with carbon dioxide. With the method of the invention, it is possible to remove from the sterol concentrates undesirable odours and flavours that hamper their use as foodstuffs as well as other harmful components and possible solvent residues. The invention also relates to a phytosterol concentrate obtained by the method and a foodstuff premix or an animal feed premix containing the same and a foodstuff or an animal feed containing said premixes. Furthermore, the invention relates to a phytosterol concentrate which is substantially free of substances causing undesirable odours and flavours and of other harmful components.

Phytosterols or plant sterols refer to naturally occurring compounds having a steroid backbone. The most common, naturally occurring phytosterols include b-sitosterol, campesterol, stigmasterol and brassicasterol.

The cholesterol-lowering effect of phytosterols has been known since the 1950s and several reports on the positive effects of the plant sterols on the cholesterol level are found in the literature of the field. The most recent reports include, for instance, that by Heineman et al., Eur. J. Clin. Pharmacol. 40 (Supp1.1), 1991, pp. 59-63, on the effect of sitostanol and sitosterol on the cholesterol absorption in the intestines by using an intestinal perfusion technique. Sitostanol was found to reduce cholesterol absorption by as much as 85%, sitosterol by 50%.

Phytosterol concentrates are manufactured industrially by various extraction and crystallization methods in which a distillate obtained from steam refining of vegetable oils and a crude soap obtained from pulp cooking are typically used as raw materials.

As regards undesirable odours and flavours and other harmful components, vegetable-oil-based, such as soybean-based, phytosterol concentrates are particularly problematic. In preparation, these concentrates retain some unsaturated fatty acids whose rancidification products cause unpleasant odours and flavours which hamper the use in foodstuffs. Typical rancidification products include, for instance, hexanal and methyl ester of octanoic acid. It has been problematic to remove impurities producing undesirable odours and flavours from the phytosterol concentrates, because the impurities tend to migrate in separation processes with the sterols.

In conventional purification methods based on extracting and crystallizing sterols, solvent residues tend to remain to some extent in the sterol concentrates, which residues are also harmful to the use of sterol concentrates as foodstuffs.

For use in pharmaceuticals and foodstuffs, a crude phytosterol concentrate has been typically purified by crystallization. For instance, U.S. Pat. No. 2,843,610 (Eastman Kodak Co.) and U.S. Pat. No. 4,420,427 (Kaukas Oy) disclose a process for crystallizing b-sitosterol from methanol/ketone/water solutions by cooling. By repeating the recrystallization procedures several times, high phytosterol concentration is achieved, which is often sufficient for pharmaceutical use. However, minor amounts of oxygen-containing compounds, which produce odour and flavour that hamper the use in foodstuffs, may remain in the crystallized phytosterol products.

U.S. Pat. No. 4,153,622 (Medipolar) discloses a process for the isolation of b-sitosterols from sterol mixtures obtained from a neutral fraction of a pulp cooking process. In this process an acetone solution containing b-sitosterol is filtered with active carbon to remove impurities, after which ethanol is added to the obtained solution and b-sitosterol is crystallized from the acetone/ethanol solution by cooling. In crystallizations and filtrations with active carbon, it is necessary to use multiple amounts of flammable and partly toxic solvents in comparison with the amount of phytosterol. After crystallizations and filtrations the solvents must be evaporated from the phytosterol product. Despite the evaporation, at least small amounts of solvents will remain in the phytosterol.

Extraction with supercritical fluids has been used for extracting sterols from plant material. One method of this kind is set forth in U.S. Pat. No. 5,252,720 (Schering Co.). In this method, the sterols are extracted from an acid-hydrolysate of plant material with a supercritical fluid, such as carbon dioxide, optionally with an organic co-solvent. The desired sterols dissolve in the supercritical fluid, from which they are recovered. The temperature used in extraction is preferably 75 to 250° C. and the pressure is 100 to 300 atm (about 101 to 304 bar). In this method, impurities causing undesirable odours and flavours cannot be separated from the sterols, since the impurities dissolve in the carbon dioxide together with the sterols. The sterol concentrate thus contains both sterols and impurities.

From EP application 0 541 853 A1 (Muiller-Extract-Company) is known use of a supercritical fluid for improving the preservability of palm oil extract and the stability of b-sitosterol contained therein and for removing unpleasant odour and flavour agents contained in the palm oil extract. b-sitosterol content in the palm oil extract is very small, about 1%. In this method, the palm oil extract is treated with pressurized, liquid carbon dioxide. The treatment is carried out in two steps, one treatment under a pressure of 75 to 350 bar and at a temperature of 20 to 90° C. and the other at a temperature of 15 to 30° C. and under a pressure of 40 to 75 bar. The described method yields a deodorized palm oil extract which has a very small b-sitosterol content (about 1%).

However, the use of pressurized fluids for purifying the phytosterol concentrates has not been reported in the literature.

### DEFINITIONS ASSOCIATED WITH THE INVENTION

In connection with the present invention, phytosterols refer to free sterols and stanols and derivatives thereof, such as esters and glycosides. In the present invention, typical phytosterols include b-sitosterol, campesterol, stigmasterol and brassicasterol.

Phytosterol concentrates refer to a phytosterol preparation which contains above-defined phytosterols and whose phytosterol content is raised. The phytosterol concentrate can be a phytosterol preparation of natural origin. The phytosterol concentrate can be based on tall oil, for instance, typically originating from a soap fraction of a pulp cooking process. The phytosterol concentrate may also originate from separation processes of phytogenic tocopherols, for instance from a distillation fraction obtained from steam refining of vegetable oils. The vegetable oil raw material is typically soybean-, rapeseed-, sunflower- or maize-based vegetable oil.

The phytosterol concentrate can be in a solid form, whereby its phytosterol content is typically over 80%, preferably 90 to 98%. The phytosterol concentrate can also be in the form of a liquid or suspension, whereby its phytosterol content is typically in the range of 20 to 80%, preferably 40 to 60%.

In connection with the present invention, the pressurized fluid refers to a food-grade, non-polar substance or mixture of substances that is gaseous at normal atmospheric pressure and that is used in a pressurized state in the present invention. The density of the pressurized fluid should be sufficient to dissolve the impurities of the sterol concentrate. To achieve sufficient dissolving properties, the minimum pressure of the fluid is typically 50 bar.

Preferably, the pressurized fluid to be used according to the invention mainly consists of carbon dioxide. In addition to carbon dioxide, it is also possible to use propane, for instance. The pressurized fluid can also contain solvents, such as alcohols with low molecular weight, for instance ethanol.

### BRIEF DESCRIPTION OF THE INVENTION

According to the present invention, it has been found that by treating a phytosterol concentrate with a pressurized fluid, typically with carbon dioxide, it is possible to remove components producing undesirable odours and flavours selectively without losing sterols with the fluid. The method enables the preparation of a solid, food-grade, pure phytosterol product without multi-stage crystallization and without flammable or toxic organic solvents.

Separation of carbon dioxide from a purified phytosterol concentrate does not require any special measures. When the method is performed on a continuous or semi-continuous basis, the carbon dioxide stream containing impurities is simply diverted from the purified phytosterol concentrate. It is also possible to allow the carbon dioxide to evaporate from the purified phytosterol.

### DETAILED DESCRIPTION OF THE INVENTION

The object of the invention is to provide a method of purifying phytosterol concentrates, in which method the phytosterol concentrate is contacted with a pressurized fluid. The temperature and pressure to be used are typically in the range of −20 . . . 180° C. and 50 to 600 bar.

The pressurized fluid is typically a food-grade, non-polar substance or mixture of substances that is gaseous at normal atmospheric pressure. The fluid to be used may also contain solvents, such as alcohols with low molecular weight, e.g. ethanol.

It is advantageous to use a pressurized fluid which mainly consists of carbon dioxide. Carbon dioxide is an environment-friendly, pure gas which does not provide additional impurities in the final product to be purified. However, it is also possible to use other food-grade gases, such as propane.

The method is suitably performed at a temperature of 10 to 75° C., preferably at 20 to 60° C., most preferably at 35 to 45° C. A suitable pressure range is 80 to 300 bar, preferably 150 to 250 bar.

Technically, the method works within the entire above-mentioned temperature and pressure range of −20 . . . 180° C. and 50 to 600 bar. The higher the working temperature, the more sterols start dissolving in the carbon dioxide, whereby the yield of sterols decreases while the purity of the phytosterol concentrate increases. At lower temperatures, the sterol losses are lower, but correspondingly, the product may contain more impurities. The lower the pressure, the lower the density of gas and the poorer its dissolving power.

The phytosterol concentrate used can be a tall-oil-based phytosterol concentrate, whereby it typically originates from a soap fraction of a pulp cooking process. It may also originate from separation processes of phytogenic tocopherols, for instance, from a distillation fraction from steam refining of vegetable oils. The raw material that is based on vegetable oil is typically soybean-, rapeseed-, sunflower or maize-based vegetable oil.

The phytosterol concentrate can be in a solid form, whereby its phytosterol content is typically over 80%, preferably 90 to 98%. The phytosterol concentrate can also be in the form of a liquid or suspension, whereby its phytosterol content is typically within the range of 20 to 80%, preferably 40 to 60%.

In the following particular embodiments of the invention, the method of the invention is described as applied to a carbon dioxide treatment.

The method is preferably performed as a semi-continuous process, whereby the phytosterol concentrate to be treated is placed in a pressure vessel and it is contacted with a continuous, pressurized carbon dioxide stream. Substances producing undesirable odours and flavours, colouring agents and possible solvent residues are released from the phytosterol concentrate with the carbon dioxide. Thereafter, the carbon dioxide stream that has been in contact with the phytosterol concentrate and that contains impurities is diverted on a continuous basis from the phytosterol concentrate. The purified phytosterol concentrate is recovered. A semi-continuous process is particularly well suited for phytosterol concentrates in solid form.

The method can also be implemented as a batch process or as a continuous process.

In one embodiment of the invention, the carbon dioxide treatment can be combined with other purification methods of phytosterols, such as crystallization and extraction methods. In that case, raw material is suitably a phytosterol concentrate, the phytosterol content of which is still low, for instance, about 5 to 50%. The carbon dioxide treatment can be carried out prior to the crystallization/extraction steps and/or thereafter. The carbon dioxide treatment removes impurities that inhibit crystallization (crystallization inhibitors), which facilitates crystallization steps to be carried out later and the yield from crystallization improves. When the carbon dioxide treatment is carried out after the crystallization/extraction steps, solvent residues originating from the extraction solvents can be removed.

In the extraction step, typical extraction solvents include hydrocarbons, such as hexane, and ketones, e.g. acetone. Typical crystallization solvents are aqueous alcohol solutions which may also contain ketones.

The carbon dioxide treatment in accordance with the invention can thus replace, completely or partly, purification steps implemented with organic solvents, and consequently, production costs can be reduced, use of organic solvents can be reduced and a higher yield of phytosterols can be achieved.

In a further embodiment of the method of the invention, the carbon dioxide treatment of the invention can be combined with the preparation of the actual phytosterol concentrate. In that case, the phytosterols are separated from the raw material used with the same fluid that is used for separating impurities. The conditions (pressure and temperature) are selected such that first is separated a phytosterol-rich concentrate with impurities, and thereafter the impurities are separated from the phytosterol concentrate. In that case, a phytosterol preparation dissolved in a supercritical fluid is used as the starting material in the method of the invention.

The last-mentioned embodiment of the invention comprises a preliminary stage in which a crude phytosterol preparation is first extracted with supercritical carbon dioxide (the pressure and temperature of the carbon dioxide both being simultaneously above the critical point of carbon dioxide) at a high pressure and at an elevated temperature. Thus, most of the phytosterol concentrate and a portion of the impurities dissolve in the carbon dioxide stream. The portion of the impurities that is insoluble in carbon dioxide can be separated in a supercritical extraction step. The carbon dioxide stream that contains as dissolved most of the phytosterols and impurities soluble in carbon dioxide is conducted through a reduction in pressure and temperature to a pressure and temperature range used in the method of the invention. The phytosterols then precipitate in the pressure vessel and the substances producing undesirable odours and flavours remain dissolved in the carbon dioxide stream, which is diverted from the purified, precipitated phytosterol concentrate.

The invention also relates to a purified phytosterol concentrate which is obtained by the method of the invention.

The invention further relates to a solid phytosterol concentrate, the phytosterol content of which is over 80%, preferably 90 to 98%, and which is substantially free of substances producing undesirable odours and flavours and of other harmful components.

The invention also relates to a phytosterol concentrate in the form of a liquid or suspension, the phytosterol content of which is 20 to 80%, preferably 40 to 60%, and which is substantially free of substances producing undesirable odours and flavours and of other harmful components.

The invention further relates to a foodstuff premix containing phytosterol concentrate of the invention and conventional foodstuff raw material. Said conventional foodstuff raw material is typically selected from the group comprising cereal, leguminous plants, milk powder and/or fruits, vegetables and berries, particularly in powder form, as well as bone, feather, fish, meat and/or rind. The raw material can also be e.g. fat or a fat mixture, sugar or a sugar mixture, a special sweetener or salt or a salt mixture.

In connection with the present invention, foodstuffs also comprise so-called dietary supplement products and products classifiable as pharmaceuticals.

The premix of the invention is typically a so called premix whereto considerable amounts of other substances are added in connection with the use. The premix can also be a so called complete mix that is not supplemented with other substances in connection with the use, possibly apart from water or other liquid.

One example of the premixes of the invention is a mineral-salt-based premix which contains the phytosterol concentrate of the invention and a mineral/minerals which is/are selected from the group of magnesium, calcium and potassium. The premix is also useful as an animal feed premix.

Another example of the premixes of the invention is a premix which contains the phytosterol concentrate of the invention and an emulsifier and optionally also a fat component. The premix can be in solid form or in the form of an oil or suspension. The premix is also useful as an animal feed premix.

The premix of the invention can also be a premix which is intended for sweets products and contains the phytosterol concentrate of the invention and conventional sweets raw material. The premix may also be a chocolate premix containing the phytosterol concentrate of the invention and conventional chocolate raw material. One example is a sugar-based premix containing the phytosterol concentrate of the invention and saccharose. The amount of the phytosterol is typically 5 to 25%, preferably 10 to 20%.

One more example of the premixes of the invention is a premix for sausage products which contains the phytosterol concentrate of the invention and conventional sausage raw material.

The premix of the invention may also be a premix intended for bakery products which contains the phytosterol preparation of the invention and conventional baking raw material. This premix is typically a cereal-based premix. One premix of this kind is a premix containing the phytosterol concentrate and flour. The flour is typically rye, buckwheat, maize or wheat flour and the premix typically contains about 5 to 25%, more preferably about 10 to 20% of phytosterol.

The premix of the invention may also be a premix intended for milk products which contains the phytosterol concentrate of the invention and conventional milk product raw material. One example is a premix intended for yoghurt products.

The amount of the purified phytosterol concentrate in the premix may vary widely depending on the composition and field of use of the premix. In general, the amount of the phytosterol is about 1 to 60% by weight, preferably about 5 to 50, more preferably about 10 to 40 and most preferably about 10 to 20% by weight.

The premix is typically prepared such that a homogenous, finely divided mixture is formed from the phytosterol concentrate and a conventional foodstuff raw material that is defined above.

The invention also relates to foodstuffs which contain the phytosterol concentrate of the invention or the foodstuff premix of the invention.

The invention further relates to premixes intended for animal feeds and containing the phytosterol preparation of the invention and conventional animal feed raw material. Additionally, the invention relates to an animal feed containing the phytosterol preparation of the invention or the animal feed premix of the invention.

The following examples describe in greater detail some embodiments of the invention. However, the invention is not restricted to the implementations described in the examples.

### EXAMPLE 1

118.1 g of soybean-based, powdery phytosterol concentrate was introduced in a tubular pressure vessel (capacity 200 ml), the upper and lower end of which was provided with a porous metal sinter. The phytosterol content of the concentrate was 96% (determined by gas chromatogrgaphy) and its phytosterol composition was as follows: 28% campesterol, 42% b-sitosterol and 26% stigmasterol. The phytosterol powder was placed in a space between the sinters at the upper and lower ends of the pressure vessel.

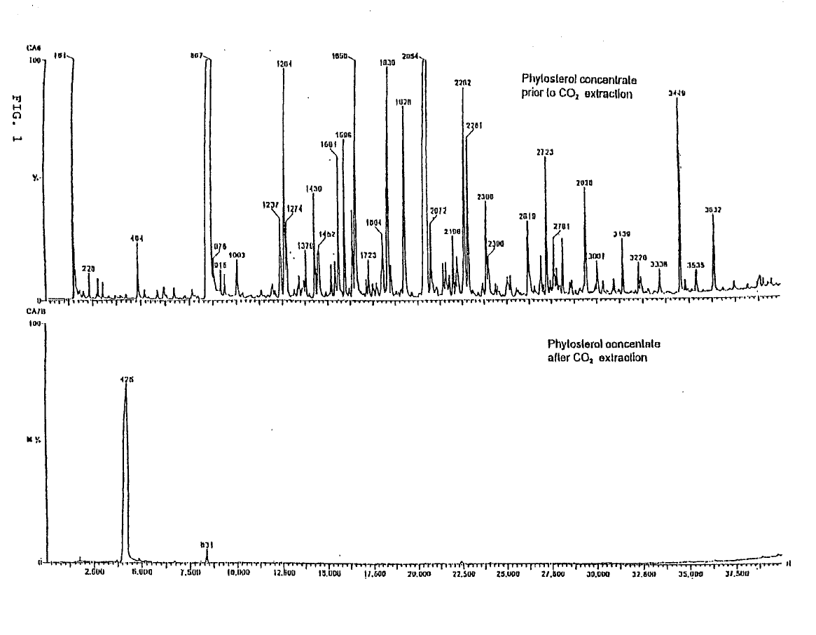

The phytosterol powder used as a starting material was yellowish grey in colour and it had a strong odour. A gas chromatographic head-space analysis showed that the powder contained tens of volatile components. Quantitatively, the main components were hexanal and methyl ester of octanoic acid (see the upper spectrum in FIG. 1, the hexanal peak at point 867 and the peak of the methyl ester of octanoic acid at point 2054).

The pressure vessel containing impure phytosterol powder was heated up to 40° C., and carbon dioxide was continuously conducted through the vessel at a pressure of 235 to 240 bar, at a rate of 6 g/min. The carbon dioxide stream that discharges from the pressure vessel was conducted through a pressure reducing valve to a glass vessel having normal atmospheric pressure, whereby the carbon dioxide discharged as gas and brownish liquid started accumulating in the vessel. During the first 50 minutes, 0.57 g of liquid was accumulated. When liquid accumulation stopped, the carbon dioxide was stopped to pass through the sterol powder. Altogether, the amount of brownish liquid extracted from the sterol powder in this manner was 1% of the weight of the phytosterol powder. All the compounds detected in the head-space analysis of the non-purified sterol powder were recognizable in a chromatographic analysis of the extract. Also the main components were the same.

It appears from FIG. 1 that the carbon dioxide treatment had succeeded in removing the harmful components from the sterol powder (see the lower spectrum in FIG. 1). As regards the odour, the purified sterol powder emitted only an extremely weak, unidentifiable odour. The purified sterol powder was white in colour.

### EXAMPLE 2

The same test arrangement was used as in Example 1.

96.9 g of the same soybean-based phytosterol concentrate as above was weighted in the space between the sinters of the pressure vessel, and liquid carbon dioxide was pumped through the pressure vessel at a temperature of 25° C. and at a pressure of 150 bar. The extract obtained was first yellowish, semisolid matter with a strong odour. When the extract had turned into a white, solid matter, the carbon dioxide was stopped to pass through the sterol powder. In this manner, 0.84 g of extract was obtained from the phytosterol powder. The phytosterol concentrate extracted with the carbon dioxide was nearly white in colour and it emitted a weak, unidentifiable odour.

### EXAMPLE 3

An impure, soybean-oil-based phytosterol concentrate was prepared first. A steam distillate, which was obtained from deodorizing step of soybean oil and which contains about 5% of phytosterols, was saponified and acidified as described in Example 1 of U.S. Pat. No. 2,843,610. The obtained oil fraction was dissolved in a boiling mixture of acetone, methanol and water, and the solution was allowed to cool to room temperature. A precipitate was crystallized from the solution and the precipitate was filtered from the mother liquor.

The obtained, solvent-containing phytosterol concentrate in the form of a yellowish precipitate is extracted by using the test arrangement described in Example 1. The extraction is performed with carbon dioxide, at a temperature of 50° C. and at a pressure of 180 bar. The product obtained is nearly white, odourless and solid phytosterol with a phytosterol concentration exceeding 96%. No significant acetone or methanol residues, nor volatile components, are detected in a gas chromatographic head-space analysis of the product.

### EXAMPLE 4

The same test arrangement is used as in Example 1. Impure phytosterol preparation is introduced in the pressure vessel, and carbon dioxide is passed through the pressure vessel at a temperature of 150° C. and at a pressure of 380 bar at most. In these conditions, most of the phytosterols and the impurities soluble in carbon dioxide dissolve in the carbon dioxide stream.

The carbon dioxide stream, which comes from the pressure vessel and which contains most of the phytosterols and impurities soluble in carbon dioxide, is passed on a continuous basis through a pressure reducing valve to a second pressure vessel having a temperature of 40° C. and a pressure of 240 bar. Thus, the phytosterols precipitate, while the carbon-dioxide-soluble impurities remain dissolved in the carbon dioxide stream. From this pressure vessel the carbon dioxide stream is still continuously forwarded through the next pressure reducing valve to a vessel having a normal atmospheric pressure. After the test, a small amount of yellowish matter, which remained insoluble in carbon dioxide (impurities insoluble in carbon dioxide), is collected from the first pressure vessel. From the second pressure vessel is collected pure white, odourless, phytosterol concentrate (a precipitated phytosterol product). Brownish, oily matter (impurities soluble in carbon dioxide) accumulate in a glass vessel having a normal atmospheric pressure.

### EXAMPLE 5

### Examples of Foodstuff Premixes

(A) Cereal-based premix

2 kg of phytosterol preparation, which is obtained according to Example 1 and which is pulverized by impact milling or jet pulverizing, is carefully mixed with 18 kg of grains. The grains can be rye grains or wheat grains husked in various ways. The mixture is ground with a grinder based on the impact milling principle and represented by the Atrex mill (Megatrex Oy, Haravalta).

Processing conditions were as follows:

| | |
|---|---|
| Upper rotor (rpm) | 3000 |
| Lower rotor (rpm) | 3000 |
| Mass flow of grains (kg/min) | 2.5 |
| Temperature of rye (° C.) | 25 |

When passing through the grinder, the grains are crushed and the phytosterol particles are ground with the grains into a homogenous phytosterol-flour mixture.

A cereal-based, phytosterol-containing foodstuff premix is obtained, which is useful for preparing bakery products, for instance.

(B) Sugar-based premix

20 g of phytosterol preparation obtained according to Example 1 and pulverized by jet pulverizing or impact milling is mixed with 180 kg of saccharose and the mixture is jet-pulverized with a dry collision grinder using a classifier (Oy Finnpulva Ab, grinder FP3P, classifier FPC15R). The carrier gas was air having a temperature of 63° C. and a feed pressure of 2.4 bar.

A sugar-based, phytosterol-containing foodstuff premix is obtained, which is useful for preparing chocolate, for instance.

(C) Sausage containing phytosterol concentrate

A sausage was prepared by using a conventional cooked sausage recipe with following ingredients:

| | |
|---|---|
| Beef-based ingredients | 30-40% |
| Pork-based ingredients | 30% |
| Water | 25% |
| Milk powder and/or | |
| potato flour, wheat flour | 5% |
| Salt | 1.5% |
| Admixtures (phosphates, etc) | 1-2% |

Phytosterol was added by replacing milk powder with a mixture of milk powder and phytosterol in which mixture the proportion of phytosterol was 20%. A product of a Finnish ring sausage type was prepared of the mixture.

### EXAMPLE 6

Preparation of an animal feed containing phytosterol concentrate

A chicken feed was prepared by using the following basic recipe of chicken feed:

| | |
|---|---|
| Starch raw material | 60-70% |
| (mainly barley) | |
| Protein component | 20-30% |
| (mainly crushed soybean) | |
| Other ingredients | 5-10% |
| minerals (mainly CaCo₃) | |
| vitamins | |
| trace elements | |
| amino acids | |
| enzymes, if any | |

The phytosterol concentrate of the invention was ground with CaCO₃ into a premix containing about 20% of phytosterol. This premix was added to other animal feed components so that the final phytosterol content of the animal feed mixture was 1%.

It is obvious to the person skilled in the art that as technology progresses the basic idea of the invention can be implemented in a variety of ways. Thus, the invention and its embodiments are not restricted to the above-described examples, but they may vary within the scope of the claims.

1. A method of purifying phytosterol concentrates, comprising contacting the phytosterol concentrate having a phytosterol content of 20 to 80% or over 80% with a pressurized fluid.

2. A method as claimed in claim 1, wherein the pressurized fluid is a food-grade, non-polar substance or mixture of substances that is gaseous at normal atmospheric pressure.

3. A method as claimed in claim 2, wherein the pressurized fluid mainly consists of carbon dioxide.

4. A method as claimed in claim 1, wherein the method is carried out at a temperature of −20° C. to 180° C. and at a pressure of 50 to 600 bar.

5. A method as claimed in claim 4, wherein the method is carried out at a temperature of 10 to 75° C. and at a pressure of 80 to 300 bar.

6. A method as claimed in claim 4, wherein the method is carried out at a temperature of 20 to 60° C. and at a pressure of 150 to 250 bar.

7. A method as claimed in claim 6 wherein the method is carried out at a temperature of 35 to 45° C.

8. A method as claimed in claim 1, wherein the phytosterol concentrate is obtained from a distillation fraction of steam refining of vegetable oils.

9. A method as claimed in claim 1, wherein the phytosterol concentrate is obtained from a soap fraction of a pulp cooking process.

10. A method as claimed in claim 1, wherein the phytosterol concentrate is in solid form.

11. A method as claimed in claim 10, wherein the phytosterol content of the phytosterol concentrate is over 80%.

12. A method as claimed in claim 10, wherein the phytosterol content of the phytosterol concentrate is 90 to 98%.

13. A method as claimed in claim 1, wherein the phytosterol concentrate is in the form of a liquid or a suspension.

14. A method as claimed in claim 13, wherein the phytosterol content of the phytosterol concentrate is 20 to 80%.

15. A method as claimed in claim 13, wherein the phytosterol content of the phytosterol concentrate is 40 to 60%.

16. A method as claimed in claim 1, wherein the method is carried out as a semi-continuous process.

17. A method as claimed in claim 1, wherein the method comprises additional steps, in which the carbon dioxide that contains impurities is separated from the phytosterol concentrate and the pure phytosterol concentrate is recovered.

18. A method as claimed in claim 1, wherein the method further comprises one or more extraction and/or crystallization steps prior to the treatment with the pressurized fluid and/or thereafter.

19. A method as claimed in claim 1, wherein a phytosterol preparation dissolved in a supercritical fluid is used as the starting material.

20. A purified phytosterol concentrate prepared by a method as claimed in claim 1.

21. A solid phytosterol concentrate, wherein the phytosterol content is over 80% and which is substantially free of substances producing undesirable odours and flavours and of other harmful components.

22. A solid phytosterol concentrate as claimed in claim 21, wherein the phytosterol content ranges from 90 to 98%.

23. A phytosterol concentrate in the form of a liquid or a suspension, wherein the phytosterol content is 20 to 80% and which is substantially free of substances producing undesirable odours and flavours and of other harmful components.

24. A phytosterol concentrate as claimed in claim 23, wherein the phytosterol content is 40 to 60%.

25. A foodstuff premix comprising a phytosterol concentrate as claimed in claim 20 and conventional foodstuff raw material.

26. A premix as claimed in claim 25, comprising said phytosterol concentrate and one or more minerals selected from the group consisting of magnesium, calcium and potassium.

27. A premix as claimed in claim 25, comprising said phytosterol concentrate and an emulsifier and optionally a fat component.

28. A premix as claimed in claim 25, wherein said conventional foodstuff raw material is conventional sweets raw material, conventional chocolate raw material, conventional sausage raw material, conventional bakery product raw material for bakery products or conventional milk product raw material.

29. A foodstuff, comprising a phytosterol concentrate as claimed in claim 20 or a foodstuff premix comprising said phytosterol concentrate and a conventional foodstuff raw material.

30. A premix for animal feeds, comprising a phytosterol concentrate as claimed in claim 20 and conventional raw material for animal feeds.

31. An animal feed, comprising a phytosterol concentrate as claimed in claim 20 or a premix for animal feed comprising said phytosterol concentrate and conventional raw material for animal feeds.

32. A premix for dietary supplement products, comprising a phytosterol concentrate as claimed in claim 20 and conventional raw material for dietary supplement products.

33. A dietary supplement product, comprising a phytosterol concentrate as claimed in claim 20 or a premix for dietary supplemental products comprising said phytosterol concentrate and conventional raw material for dietary supplement products.

34. A premix for pharmaceuticals, comprising a phytosterol concentrate as claimed in claim 20 and conventional raw material for pharmaceuticals.

35. A pharmaceutical, comprising a phytosterol concentrate as claimed in claim 20 or a premix for pharmaceuticals comprising said phytosterol concentrate and conventional raw material for pharmaceuticals.
